(19) United States
(12) Patent Application Publication
(10) Pub. No.: US 2019/0151350 A1
(43) Pub. Date: May 23, 2019

(54) DEPOLYMERIZED HOLOTHURIAN GLYCOSAMINOGLYCAN COMPOSITION AND PREPARATION METHOD AND APPLICATION THEREOF

(71) Applicants: Suzhou Yi-hua Biomedical Technology co., Ltd, Suzhou (CN); Suzhou Ronnsi pharma co., Ltd, Suzhou (CN)

(72) Inventors: YING-YING ZHU, Suzhou (CN); YONG-SHENG JIN, Suzhou (CN); LIN-LIN WANG, Suzhou (CN); WU CHEN, Suzhou (CN); YI JIANG, Suzhou (CN); YI-MING YAO, Suzhou (CN)

(73) Assignee: Suzhou Yi-hua Biomedical Technology co., Ltd

(21) Appl. No.: 15/818,678

(22) Filed: Nov. 20, 2017

(51) Int. Cl. A61K 31/726; C08B 37/00; A61P 35/00

(52) U.S. Cl. CPC A61K 31/726; A61P 35/00; C08B 37/0063

(57) ABSTRACT

The present invention discloses a depolymerized holothurian glycosaminoglycan composition and a preparation method and application thereof. The composition comprises one or more of depolymerized holothurian glycosaminoglycans with weight-average molecular weight between 2000 Da and 12000 Da. The preparation method of the depolymerized holothurian glycosaminoglycan composition comprises the steps of extracting and purifying holothurian glycosaminoglycan, depolymerizing the holothurian glycosaminoglycan and the like. Anti-tumor studies show that the depolymerized holothurian glycosaminoglycan composition can remarkably inhibit tube formation of human umbilical vein endothelial cells in vitro and inhibit metastasis of melanomas and breast cancer in vivo. With its excellent anti-cancer properties, depolymerized holothurian glycosaminoglycan composition can be used as pharmaceuticals, nutraceuticals and other health products.

### FIELD

The present invention relates to a depolymerized holothurian glycosaminoglycan composition with a weight-average molecular weight range between 2000 Da and 12000 Da, a preparation method thereof, and application of the depolymerized holothurian glycosaminoglycan composition to prevention and treatment of cancer.

### BACKGROUND

Cancer is the second leading cause of death globally, and was responsible for almost 10 million deaths annually. Globally, nearly 1 in 6 deaths is due to cancer. Currently, standard treatment includes surgery, radiotherapy, chemotherapy and immunotherapy. Chemotherapy, as one of the most common treatment for cancer patient, can cause seriously adverse reaction and drug resistance. Chemotherapy can damage or stress cancer cells, which may then lead to cancer cell death if apoptosis is initiated. The seriously side effects of chemotherapy is its poor selectivity. It can cause damage to normal cells as well, especially for those cells that divide rapidly and are thus sensitive to anti-mitotic drugs: cells in the bone marrow, digestive tract and hair follicles. Therefore, research and development on novel drug capable of being co-administrated with anti-tumor drugs to reduce side effect, improve selectivity, enhance sensitivity and increase efficacy are of great significance.

Holothurian glycosaminoglycan (HG or HGAG) is a sulfated glycosaminoglycan containing a branched fucose side chain and is extracted from holothurian, and has a similar but significantly different structure from heparin. Its structure is shown below. HG monosaccharide composition comprises galactosamine, glucuronic acid, fucose and sulfate groups. The glucuronic acid and the galactosamine are connected through β(1-3) and β(1-4) glucosidic bonds. This disaccharide repeating unit is similar to that of chondroitin sulfate E from mammals. The sulfated fucose is a side chain attached to the main chain (Vieira R P et al., J. Biol. Chem., 1991, 266:13530-13536). HGs prepared from different source of holothurian and by different methods are structurally different (Ken-ichiro Y et al., Tetrahedron Letters, 1992, 33:4959), with difference in 4-position and 6-position sulfation of galactosamine in the main chain and difference in sulfation of the fucose side chain.

In vivo and vitro assays have demonstrated that HG has strong anticoagulation and antithrombotic effects. Although HG and heparin are both glycosaminoglycans and have similar biological activity, their structures are different and their mechanism of anticoagulation is different. Heparin binds to Antithrombin III and then inhibits protease Xa and IIa activity while HG acts on multiple anticoagulation pathways and has low risk of bleeding.

Heparin is known for its high anticoagulation activity among all the currently discovered natural products. Its anti-inflammation and anti-tumor effects have been proved in various preclinical and clinical studies when being used alone or combined with other drugs. Low-molecular-weight heparin such as Dalteparin has already been extensively used in the treatment of a variety of malignant conditions by combining with other anti-tumor drugs. Often high dose is required to show significant anti-cancer activity. However, due to its high anticoagulation activity, bleeding risk will increase when high dose is used. Therefore research efforts on developing heparin or heparin like molecules with high anti-cancer activity and low anticoagulation activity has been carried out in academic and pharmaceutical companies for years, but with limited success.

Holothurian glycosaminoglycan has lower anticoagulation activity compared to Heparin. The inventor discovered that depolymerized holothurian glycosaminoglycan (dHG) has a much lower anticoagulation activity than undepolymerized holothurian glycosaminoglycan. Therefore dHG may offer similar anti-cancer properties with much less bleeding risk. Prior to the present invention, there is no report on treatment of cancer with dHG. dHG could offer significant benefits for cancer patient.

### SUMMARY

Objectives: The primary objective of the present invention is to provide a depolymerized holothurian glycosaminoglycan composition with weight-average molecular weight between 2000 Da and 12000 Da, which can be used for treatment cancer alone or used with other anti-tumor drugs. Another objective of the present invention is to provide a simple and efficient method to prepare the depolymerized holothurian glycosaminoglycan composition. Both in-vitro and in-vivo anti-tumor experiments prove that depolymerized holothurian glycosaminoglycan composition has significant anti-tumor activity, thus providing a new composition for cancer treatment.

The present invention provides a depolymerized holothurian glycosaminoglycan composition that comprises one or more of depolymerized holothurian glycosaminoglycans with weight-average molecular weight between 2000 Da and 12000 Da.

A preparation method of the depolymerized holothurian glycosaminoglycan composition comprises the following steps:

(1) extracting holothurian glycosaminoglycan from holothurian and purifying the holothurian glycosaminoglycan; and

(2) depolymerizing the extracted holothurian glycosaminoglycan.

Preferably, according to the preparation method of the depolymerized holothurian glycosaminoglycan composition, the holothurian in step (1) is selected from but not limited to one or more of Holothuria arenicola, Holothuria atra, Holothuria leucospilota, Holothuria scabra, Holothuria nobilis, Pearsonothuria graeffei and Actinopyga mauritiana which all belong to echinodermata family. Holothuria leucospilota is native to the south China sea with its abundance. Its low price and high glycosaminoglycan content is an ideal raw material for holothurian glycosaminoglycan. However the holothurian glycosaminoglycan can be prepared from one or more of other holothurian in echinodermata family.

The extraction in present invention in step (1) comprises grinding holothurian, enzymatic hydrolysis, fractional precipitation, and resin absorption and separation, to obtain crude holothurian glycosaminoglycan; enzymes used include one or more of alkaline protease, neutral protease, pancreatin and papain; and fractional precipitation by organic solvents such as methyl alcohol, ethanol, isopropyl alcohol or acetone, and resin absorption and separation by ionic exchange resin separation and fractional precipitation.

Preferably, according to the present invention in step (1) comprises resin adsorption and fractional elution, hydrogen peroxide decolorization and endotoxin removal, to obtain pure holothurian glycosaminoglycan product.

Ion exchange resin can be cationic exchange resin or anion exchange resin; and elution is fractional elution using water and salt solution with salt concentration of 1-20% (w/w).

Preferably, according to the present invention, depolymerization of holothurian glycosaminoglycan in step (2) comprises hydrogen peroxide depolymerization for 2-48 h at temperature of 20-80° C. in an acid environment, to obtain the depolymerized holothurian glycosaminoglycan composition with weight-average molecular weight between 2000 Da and 12000 Da.

The strength of the hydrogen peroxide is 0.1-30% (v/v); acid used can be formic acid, acetic acid, propanoic acid, hydrochloric acid or sulfuric acid; and the strength of the acid is 0.1-20% (v/v).

Preferably, according to the preparation method of the depolymerized holothurian glycosaminoglycan composition, the step (2) of depolymerization of the extracted holothurian glycosaminoglycan comprises fractional precipitation with different concentration of sodium chloride and alcohol, and then freeze drying or vacuum drying to achieve the depolymerized holothurian glycosaminoglycan composition with a dispersity lower than 1.5.

The present invention provides a healthcare product composition comprises the depolymerized holothurian glycosaminoglycan composition and acceptable carriers in the healthcare product.

The present invention also provides a pharmaceutical composition comprises the depolymerized holothurian glycosaminoglycan composition and pharmaceutically acceptable carriers.

The present invention further provides a drug or drug composition of the depolymerized holothurian glycosaminoglycan for preventing and treating cancer. The drug or drug composition can be depolymerized holothurian glycosaminoglyca alone or combined with other anti-cancer drug for prevention or treatment of cancer.

The process to obtain depolymerized holothurian glycosaminoglycan with weight-average molecular weight between 2000 Da and 12000 Da is optimized, so reliable and scalable production can be achieved. Depolymerized holothurian glycosaminoglycan is further characterized by molecular weight distribution, optical rotation, sulfate to carboxylate ratio, nuclear magnetic resonance spectrum and other analytical methods.

The present invention also disclosed in-vitro and in-vivo study of the depolymerized holothurian glycosaminoglycan. The depolymerized holothurian glycosaminoglycan (dHG) can significantly inhibit angiogenesis formation in human umbilical vein endothelial cells (HUVEC). It also inhibit lung metastasis of melanomas and breast cancer in mice, and its inhibition efficacy is dose dependent.

### BRIEF DESCRIPTION OF THE DRAWINGS

FIG. 1 is molecular weight distribution of depolymerized holothurian glycosaminoglycan (dHG).

FIG. 2 is ¹H-NMR of holothurian glycosaminoglycan (HG) and the depolymerized holothurian glycosaminoglycan (dHG).

FIG. 3 is HUVEC tube formation result.

FIG. 4 is a lung metastatic tumor foci count histogram from a mice breast cancer model.

FIG. 5 is a lung metastatic tumor coverage histogram from a mice breast cancer model.

### DETAILED DESCRIPTION OF THE DISCLOSURE

The present invention is further described in the following embodiments, but is not limited to these embodiments.

### Embodiment 1: Extraction and Purification of Holothurian Glycosaminoglycan (HG)

1. 9.0 L of water was added to 5 kg of dried Holothurian. After 24 hours, the Holothurian was minced. Additional 11.0 L of water was added, then pancreatin 20 g was added to the mixture. The digestion lasted 6.5 hours at 50° C. and pH 7.0-8.0. The solution is filtered and 1 kg of VPOC1074 resin was added to the above filtrate, overnight resin absorption at room temperature and pH 8.0-9.0. The resin was filtered and washed with water, then washed 3 times with 2 L of 6.5% NaCl (w/w). The desired Holothurian glycosaminoglycan was eluted 3 times with 2 L of 10.5% NaCl (w/w). The combined elution (6.0 L) was precipitated with 1.2 volume of 95% ethanol (7.2 L) at 4° C. overnight. The precipitate was centrifuged at 4000 rpm for 10 min, then dehydrated twice with 95% ethanol, dried to obtain the HG.

2. The obtained holothurian glycosaminoglycan crude product is dissolved in 20 times of water by weight. pH is adjusted with 4M NaOH solution to 10, then hydrogen peroxide is added to reach concentration of 3% (v/v) for decolorization, and the reaction mixture is stirred for 2 h at room temperature. 1% NaCl and one volume equivalent of ethyl alcohol are added to the reaction mixture, the solid is isolated and re-dissolved in 10 times of water by weigh, and followed by precipitation with 1% NaCl and ethyl alcohol. The precipitation process is repeated twice. Solid obtained is washed twice with ethyl alcohol, and then vacuum dried at 45° C. to obtain 22.5 g of a holothurian glycosaminoglycan pure product.

### Embodiment 2: Preparation of Depolymerized Holothurian Glycosaminoglycan (dHG)

1. Preparation of dHG: 20 g of the holothurian glycosaminoglycan pure product prepared in embodiment 1 was dissolved in 160 mL of water, then 19.0 mL of acetic acid and 60.0 mL of hydrogen peroxide were added. The reaction mixture is stirred for 22 h at 60° C., then cooled in an ice bath. 4M of NaOH solution is added to adjust pH to 9.5, then 2 times by volume of ethanol is added, the solid is isolated by centrifugation to obtain the depolymerized holothurian glycosaminoglycan crude product.

The crude dHG product is dissolved with 200 mL of water, then 3% by weight of NaCl is added. The dHG is precipitated out by adding 1 volume of ethyl alcohol. The precipitation is repeated once with 2% NaCl and once with 1% NaCl. The solid obtained is washed twice with ethyl alcohol, and then vacuum dried to obtain 18.0 g of depolymerized holothurian glycosaminoglycan (dHG).

2. Physicochemical Properties of dHG

2.1 Molecular Weight Distribution

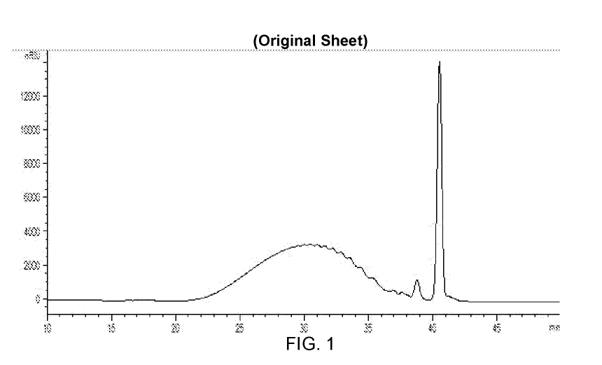

The depolymerized holothurian glycosaminoglycan (dHG) is subjected to HPGPC analysis. Two columns are used for molecular weight analysis TSKgel G2000SW (7.8 mm×30 cm, 5 μm, product number: T08542) and TSKgel G3000SW (7.8 mm×30 cm, 5 μm, product number: T10407-02T). The weight-average molecular weight measured for dHG is 10092 Da, and dispersity is 1.3. Refer to FIG. 1 (The peak at 40.5 min is solvent peak in FIG. 1).

2.2 Optical rotation: Following Chinese Pharmacopoeia (2010), A WZZ-IS type automatic polarimeter with a sodium light source (λ₅₈₉ₙₘ) is used for dHG optical rotation measurement. The sample tube is 1 dm. The optical rotation for dHG is −56.5° C. while HG is −60.5° C. Results indicated that the depolymerization process of the present invention did not change structure significantly.

2.3 Ratio of sulfate to carboxylate: Titration method with a conductivity meter was used to measure sulfate to carboxylate ratio. The ratio of sulfate to carboxylate of dHG is 3.84. This value is close to parent HG 3.55. Result demonstrated that deploymerization process in present invention did not reduce sulfate content.

2.4 ¹H-NMR

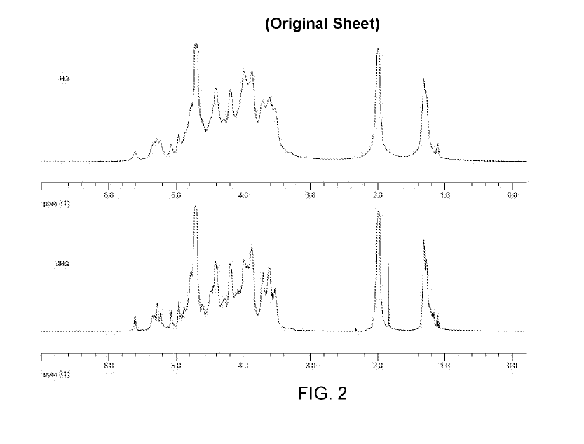

¹H-NMR spectra of holothurian glycosaminoglycan (HG) and depolymerized holothurian glycosaminoglycan (dHG) are compared and they are shown in FIG. 2. No significant difference is observed other than that ¹H NMR peaks of HG is slightly broader because of it higher molecular weight. This demonstrated that there is no or limited structure change of depolymerized HG. δ1.33 ppm is the characteristic peak of methyl group from fucose ring, and δ1.92 ppm is the characteristic peak of acetyl methyl group from galactosamine. The main difference for different holothurian glycosaminoglycan is sulfation in 4-position and 6-position of galactosamine in the main chain, and also difference of sulfation profile of the fucose side chain. A single peak at δ4.22 ppm indicated that galactosamine of holthuria leucospilota glycosaminoglycan has 6-position sulfation rather than 4-position sulfation. This is consistent with what was reported in the literature (Fen Huizeng etc., Acta Pharmaceutica Sinica, 1983, 18(3):203). The side chain fucose group has about 15% of 2,4-disulfate group (δ5.65 ppm characteristic peak), about 30% of 3,4-disulfate group (δ5.26 ppm and 4.90 ppm characteristic peaks) and about 20% of 4-disulfate group (δ5.34 ppm and 5.35 ppm characteristic peaks). Other than the above reported characteristic peaks, two other peaks at δ5.10 ppm and δ5.00 ppm at low field, with chemical shift values slightly lower than that of the reported in the literature, indicated that 2-position or 3-position sulfate group monosubstitution on the holothurian glycosaminoglycan.

2.5 Anticoagulation Activity

Sheep plasma activity and chromogenic assay of anti-Xa activity of the depolymerized holothurian glycosaminoglycan (dHG) are measured and compared to that of Dalteparin (a low molecular weight heparin). The result is presented in Table 1.

TABLE 1
Anticoagulation activity

| | dHG 10092 | Dalteparin |
|---|---|---|
| Anti-Xa | 1.1 | 160 |
| Sheep Plasma Activity | 6 | 60 |

The anti-Xa activity of dHG is only 1/160 of Dalteparin. The big risk of using Dalteparin is bleeding, especially at high dose for cancer patient. Low anticoagulant activity could reduce the bleeding risk significantly. Therefore much higher dose of dHG can be used in clinical.

### Embodiment 3: Acute Toxicity of the Depolymerized Holothurian Glycosaminoglycan (dHG)

1. Materials

dHG10092 (prepared with the method according to embodiment 2 of the present invention); ICR mice.

2. Method

ICR mice are randomly divided into two groups (ten in each group, half male and half female), and are injected with 4 g/kg and 2 g/kg of dHG10092 respectively through caudal veins.

3. Results

For the 4 g/kg dose group, part of the mice suffer from convulsion, dyspnea, abdominal respiration, righting reflex loss, uracratia and the like after caudal vein administration. These symptoms are relieved 3 min after administration, but spontaneous activity is obviously less than that of a control group.

For the 2 g/kg dose group, no obvious abnormal symptom observed after caudal vein administration. The weight of mice in both groups reduced after administration. For the 4 g/kg dose group, the weight of mice is significantly different from that of a solvent control group (P<0.05) one day after administration. The weight of mice increased two days after administration. The weight of mice in each group has no obvious difference from that of the solvent control group after two days. During an observation period, there is neither mouse dead nor obvious abnormity in each group. Observation ended 14 days after the administration. Anatomical analysis is conducted after mice are sacrificed with CO₂, and no macroscopic abnormity observed in main organs of both groups. These results demonstrated that the dHG 10092 provided by the present invention is safe up to 4 g/kg in mice.

### Embodiment 4: Study on Influences of the Depolymerized Holothurian Glycosaminoglycan (dHG) on Tube Formation of HUVECs

1. Materials

Two compounds were used in this study: depolymerized holothurian glycosaminoglycan (dHG10092) having weight-average molecular weight of 10092 Da and cabozantinib

Cell strain: primary HUVECs was purchased from Allcells company.

Reagents: endothelial cell basal culture medium (Allcells, Cat#HUVEC-004B), endothelial cell complete medium (Allcells, Cat#HUVEC-004), Geltrex LDEV-Free Reduced Growth Factor Basement Membrane Matrix (without phonol red) (GIBCO, cat#A1413202), 0.25%. Trypsin-EDTA (GIBCO, Cat#25200), fetal calf serum (GIBCO, Cat#10099141), penicillium-streptomycete double-antibody solution (GIBCO, Cat#15140-122), and DMSO(Sigma, Cat#D2650).

2. Method:

2.1 Samples preparation: cabozantinib is dissolved in DMSO to obtain a 20 mM of stock solution. The stock solution is further diluted 200 times in DMSO to generate final cabozantinib stock solution. The depolymerized holothurian glycosaminoglycan dHG10092 is dissolved in 0.9% NaCl to obtain a 20 mM of stock solution. The stock solution is further diluted 50 times to obtain the 400 μm final dHG stock solution. When the drugs were added to HUVEC cells, dHG stock solution was further diluted to 200 μm, 100 μm, 50 μm, and 25 μm with basal culture medium.

2.2 Tube formation experiment: 50 μL/pore Geltrex gel is added to a 96-well plate, incubated for 1 h in an incubator at 37° C., and the gel is solidified. When HUVEC cell grow to 80% confluence (cultured with standard medium. One day before the experiment, the cells were starved overnight with 2% FBS basal culture medium), cells were digested with pancreatin and collected after centrifugation. The cells were re-suspended in basal culture medium (containing 2% FBS) and counted. Cell suspension is further diluted to 2×10⁵ cells/ml with the basal culture medium (containing 2% FBS), and then drugs stock solution (dHG and cabozantinib) were added to the cell suspension with ratio of 1:1. 100 μL of cell suspension is added to each well in the 96-well plate (lx 10⁴ pieces/well) coated with the Geltrex gel. Multi-holes inspection was performed. Photos were taken after 4 hours culture, and the number of tube formed is counted. Compound toxicity is determined by CTG method. A dose-effect diagram is made with GraphPad Prism software.

3. Results

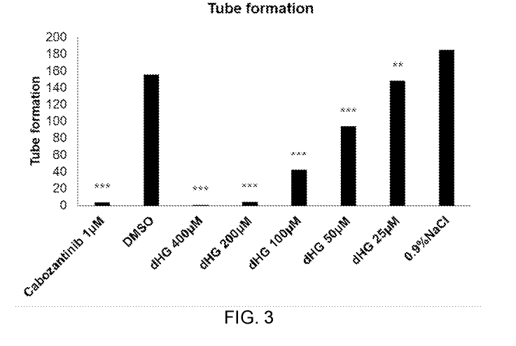

Results indicated that dHG10092 had remarkable inhibiting effect on HUVEC tube formation when concentration reached 50 μM, and the inhibition is dose dependent. Statistic analysis of tube formation is shown in FIG. 3.

### Embodiment 5: Influences of Depolymerized Holothurian Glycosaminoglycan (dHG) on Murine B16F10 Melanoma Experimental Metastasis Model

1. Materials

1.1 Animal and Cells

C57BL/6 mice (female, 16-20 g), provided by Beijing Vital River Laboratory Animal Technology Co., Ltd.

1.2. Drugs

dHG10092 (prepared with the method according to embodiment 2) and Dalteparin (Pfizer).

1.3. Reagents

DMEM (GiBCO company from the US), phosphate buffer (PBS), calf serum, trypsin, EDTA, formaldehyde, normal saline and sodium bicarbonate.

1.4. Instruments

Leica inverted fluorescence microscope, precise pipettor, fully-automatic high-pressure sterilization pan, bechtop, ultra-low-temperature refrigerator, CO₂ incubator, pure water filter, electronic scale, desk type electrothermal blowing dry box, refrigerator, liquid nitrogen container, centrifugal machine, pH meter and injector.

2. Method

2.1 Preparation of Cell Suspension

Routine culture is conducted on B16F10 tumor cells. Cell passage is conducted in cell culture bottles according to a ratio of 1:10 4-5 days before experiment, so as to avoid incomplete confluence caused by cell overgrowth, wherein each culture bottle contains about 6-8×10⁶ cells. Cells in a logarithmic phase are collected, culture solution discarded, washed with PBS. 1 mL of 0.25% pancreatin-0.02% EDTA digestive solution is added, the solution is placed in a cell incubator, and after 1-3 min, the culture bottle is slightly shaken to make the cells detached. 10 mL of DMEM is added, the cells are blown with the pipette to obtain single-cell suspension, and the single-cell suspension is transferred to a 50 mL polypropylene centrifugal tube. Cells were collected by centrifugation (1200 rpm×10 min), then washed twice with PBS. The cells were counted with trypan blue, and cell concentration was adjusted to be 2.5×10⁶/ml.

2.2 Copying of a B16F10 Experimental Tumor Metastasis Model

C57BL/6J mice were randomly divided into 3 groups based on their body weight and received a single dosed subcutaneously with vehicle saline, dHG10092 at 20 mg/kg, Dalteparin at 20 mg/kg, respectively. Then these mice were intravenously implanted with the B16F10 cell suspension (2×10⁵ cells/mice). Mice were observed daily and body weights were recorded three times per week during the experiment. At day 20, mice were euthanized Lungs were collected, weighed, photos were taken and then fixed in 10% neutral for metastatic nodule count.

3. Results

3.1 Weight

During the experiment, no obviously body weight change was observed.

3.2 Lung Weight

The lung weight of the drug group and the lung weight of the saline group are significantly different. The dalteparin group inhibited the increase of lung weight significantly, and dHG10092 provided by the present invention also inhibited the increase of lung weight. The inhibition rate of dalteparin reacheed 83.6%, and the inhibition rate of dHG10092 reached 55.7%.

3.3 Lung Tumor Node Quantity Compared with the control group (normal saline group), the tumor node quantity of the dalteparin group and the tumor node number of the dHG10092 group are significantly lower. The lung tumor node quantities of the control group (normal saline group), the dalteparin group and the dHG10092 group are 99, 17 and 34 respectively.

The results indicated that the prepared depolymerized holothurian glycosaminoglycan dHG10092 can significantly inhibit tumor metastasis when used alone at 20 mg/kg, and it has no affect on mice weight.

### Embodiment 6: Influences of Depolymerized Holothurian Glycosaminoglycan (dHG) on Orthotopic 4T1-Murine Mammary Carcinoma Model

1. Materials

1.1 Experiment Animal and Cells

BALB/C mice (female, 6-8 weeks old, 16-20 g), provided by Beijing Vital River Laboratory Animal Technology Co., Ltd.

1.2. Drugs:

dHG10092 (prepared with the method according to embodiment 2 of the present invention); Cisplatin (Qilu Pharmaceutical Co., Ltd); Dalteparin (Pfizer).

1.3. Experiment Reagents:

DMEM (GiBCO company from the US), phosphate buffer (PBS), calf serum, trypsin, EDTA, formaldehyde, normal saline and sodium bicarbonate.

1.4. Instruments

Leica inverted fluorescence microscope, precise pipette, fully-automatic high-pressure sterilization pan, bechtop, ultra-low-temperature refrigerator, CO₂ incubator, pure water filter, electronic scale, desk type electrothermal blowing dry box, refrigerator, liquid nitrogen container, centrifugal machine, pH meter and injector.

2. Method

2.1 Cell Culture

4T1 cells are cultured with RPMI-1640 culture solution, wherein the concentration of added fetal calf serum is 10%, a penicillin/streptomycin double-antibody is diluted in the culture solution based on the ratio of 1:100, and the cells are cultured in a thermostatic incubator which contains 5% CO₂ and has a saturation temperature of 37° C. The cells are subjected to passage every other 2-3 days based on the ratio of 1:3-1:5.

2.2 Condition of Mouse Pulmonary Metastasis Foci when Orthotopic Tumors are Removed

BALB/C mice are randomly divided into 7 groups, and a fourth fat pad of the breast is inoculated with 1×10⁵ 4 T1 cells. Then 20 mg/kg, 40 mg/kg and 80 mg/kg dHG10092 and 4 mg/kg dalteparin are injected to each group respectively. Mice were observed daily. Body weights and tumor volumes were recorded three times per week during the experiment. Breast tumors are removed after continuous administration for 12 days, and then 4 mg/kg cisplatin is adimistrated to each group from the 13th thy, once in every four days. On the 30th day, anesthesia is conducted, the mice are sacrificed, lungs and spleens are taken out, washed and weighted. Photos were taken and lung samples were fixed in 10% neutral for metastatic nodule count and HE staining

3. Experiment Results

3.1 Weight

The weights of all mice treated with cisplatin (alone or in a combination) reduced, indicating that cisplatin can cause weight reduction.

3.2 Tumor Weight

Different groups have no obvious difference in primary tumor growth, however, compared with the cisplatin group, the tumor weight of the cisplatin and dHG10092 combination group is lower.

3.3 Lung Weight and Spleen Weight

Compared with a negative control group, the group treated with cisplatin (alone or combined with dHG10092) has much lower lung weight and spleen weight, indicating that both treatment groups can inhibit tumor metastasis and diffusion of bone marrow cell relevant to tumors.

3.4 Lung Metastasis Quantification

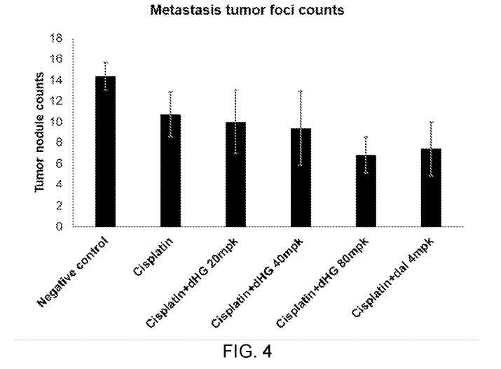

Compared with the group using cisplatin alone, combination of dHG10092 and cisplatin can remarkably reduce the lung tumor node quantity. It is dose-dependent and the lung tumor node quantity of the 80 mg/kg group is much less than that of the cisplatin independent group (P<0.05), as shown in FIG. 4.

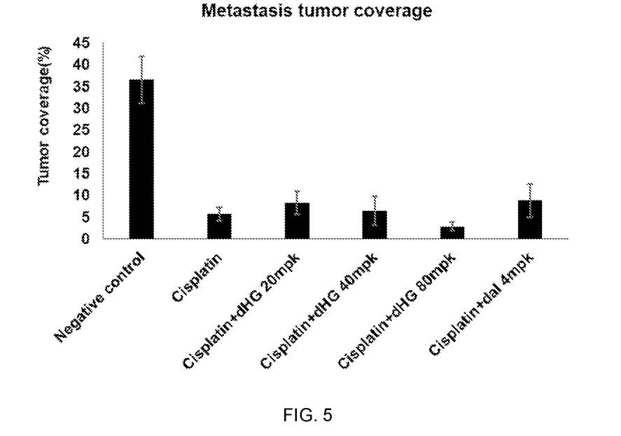

HE staining and lung tumor area quantification results indicated that all cisplatin treatment groups (using cisplatin independently or with the prepared dHG10092) can remarkably reduce the lung tumor area range, the dHG10092 provided in the present invention shows a dose-effect relationship, and the 80 mg/kg dose group has the best treatment effect. As shown in FIG. 5.

The above experiment results indicated that by combining depolymerized holothurian glycosaminoglycan dHG10092 with cisplatin, tumor metastasis can be significantly inhibited, and is dose dependent. 80 mg/kg dose group gave the best result. No adverse reaction is observed.

What is described above is merely the preferred embodiments of the present invention, and it should be noted that numerous improvements and modifications can be made by those skilled in the art without deviating from the principles of the present invention, and these improvements and modifications should also be viewed to be within the scope of the present invention.

1. (canceled)

2. A preparation method of the depolymerized holothurian glycosaminoglycan composition, characterized by comprising the following steps:
(1) extracting holothurian glycosaminoglycan from holothurian and purifying the holothurian glycosaminoglycan; and
(2) depolymerizing the extracted holothurian glycosaminoglycan,
wherein the step of depolymerizing the extracted holothurian glycosaminoglycan in step (2) comprises conducting hydrogen peroxide depolymerization reaction for 2-48 h at the controlled temperature of 20-80° C. in an acid environment, and depolymerizing to obtain the depolymerized holothurian glycosaminoglycan composition with weight-average molecular weight smaller than 12000 Da.

3. The preparation method of the depolymerized holothurian glycosaminoglycan composition according to claim 2, characterized in that the holothurian in step (1) comprises one or more of Holothuria arenicola, Holothuria atra, Holothuria leucospilota, Holothuria scabra, Holothuria nobilis, Pearsonothuria graeffei and Actinopyga mauritiana which all belong to echinodermata.

4. The preparation method of the depolymerized holothurian glycosaminoglycan composition according to claim 2, characterized in that the step of extracting the holothurian glycosaminoglycan from the holothurian in step (1) comprises conducting grinding, enzymolysis, fractional precipitation and separation of the holothurian, so that a holothurian glycosaminoglycan crude product is obtained; the enzymes for enzymolysis include one or more of alkaline protease, neutral protease, pancreatin and papain; and precipitation is conducted by means of one or more of organic solvents methyl alcohol, ethanol, isopropyl alcohol or acetone, and separation comprises ion exchange resin separation and metal salt fractional precipitation.

5. The preparation method of the depolymerized holothurian glycosaminoglycan composition according to claim 2, characterized in that the concentration of the hydrogen peroxide is 0.1-30%; acid used can be formic acid, acetic acid, propanoic acid, hydrochloric acid or sulfuric acid; and the concentration of acid is 0.1-20%.

6. The preparation method of the depolymerized holothurian glycosaminoglycan composition according to claim 2, characterized in that the step (2) of degrading the extracted holothurian glycosaminoglycan comprises conducting fractional precipitation by means of sodium chloride with different concentrations so as to achieve desalination, and then conducting freeze drying or vacuum drying, so that the depolymerized holothurian glycosaminoglycan composition with a dispersity lower than 1.5 is obtained.

7. (canceled)

8. (canceled)

9. (canceled)

10. (canceled)

11. A holothurian glycosaminoglycan composition made by the preparation method according to any one of claims 2-6.

12. A healthcare product composition, characterized by comprising the depolymerized holothurian glycosaminoglycan composition according to claim 11 and carriers acceptable in healthcare industry.

13. A pharmaceutical composition, characterized by comprising the depolymerized holothurian glycosaminoglycan composition according to claim 11 and pharmaceutically acceptable carriers.

14. An application of the depolymerized holothurian glycosaminoglycan composition according to claim 11 for preventing and treating cancer.

15. The application of the depolymerized holothurian glycosaminoglycan composition to preparation of the drugs or the healthcare products for preventing and treating cancer according to claim 14, characterized by treating cancer alone or combining with other anti-tumor drugs.
